(19) United States
(12) Patent Application Publication
(10) Pub. No.: US 2025/0010053 A1
(43) Pub. Date: Jan. 9, 2025

(54) DEVICE SET DESIGNED FOR PCNL SURGERY

(71) Applicant: BECTON, DICKINSON AND COMPANY, Franklin Lakes, NJ (US)

(72) Inventor: Xin Wen, Shanghai (CN)

(21) Appl. No.: 18/891,346

(22) Filed: Sep. 20, 2024

Related U.S. Application Data
(63) Continuation of application No. 17/000,289, filed on Aug. 22, 2020, now Pat. No. 12,109,382.

(30) Foreign Application Priority Data
Aug. 23, 2019 (CN) 201910783544.2

(51) Int. Cl. A61M 39/02; A61B 17/34; A61B 50/30; A61M 1/00; A61M 25/00; A61M 25/02; A61M 25/09; A61M 25/10; A61M 29/00; A61M 39/22

(52) U.S. Cl. CPC A61M 39/0247; A61B 17/34; A61B 50/30; A61M 1/69; A61M 25/02; A61M 25/09; A61M 25/10; A61M 29/00; A61M 39/22; A61B 2017/3413; A61B 2050/3008; A61M 1/84; A61M 25/002; A61M 2039/229

(57) ABSTRACT

A device set designed for PCNL surgery includes a puncture needle, a guidewire, a dilator, a balloon catheter, a sheath, and a drainage catheter which are all placed in a single package. A tip length of the dilator is substantially the same as a tip length of the balloon catheter. The device set may include a puncture needle, a guidewire, a plurality of dilators having different sizes, a sheath, and a drainage catheter, which are all placed in a single package, wherein all the dilators have substantially the same tip length.

This application is a continuation of U.S. patent application Ser. No. 17/000,289 file on Aug. 22, 2020, which claims the benefit of Chinese Patent Application No. 201910783544.2, filed Aug. 23, 2019, the disclosures of which are incorporated herein by reference.

### TECHNICAL FIELD

The present disclosure relates to the percutaneous nephrolithotomy (PCNL) surgery field, in particular, to a device set designed for PCNL surgery.

### BACKGROUND

Percutaneous nephrolithotomy, commonly referred to as “drilling holes and removing stones,” is a very effective method of removing and crushing kidney stones. Almost all kidney stones can be removed by percutaneous nephrolithotomy. Successful rate of renal pelvic stone removal is 96-98%, and successful rate of ureteral stone removal is 85%.

For PCNL surgery, there are two common ways to create a nephrostomy tract for removing calculi (i.e., a stone removal path). One way is using balloon dilation catheter to create a tract; and another way is using balloon dilation catheter to create a tract. Current products, especially a balloon dilation product, only includes part of required components. Thus, the physician needs to acquire several packages in order to get all required components, which is very inconvenient.

Moreover, this will also create waste in two aspects. Firstly, a total cost for several separate packages is higher than that for one package. Secondly, different packages will include the same components or extra (undesired) components, which will not be used during surgery (and it is not allowed to sterilize these components and reuse them). Physicians have to discard these components, which will also create waste.

For example, in the method of balloon dilation, physicians have to purchase a balloon dilation catheter set and a fascia dilation device set currently commercially available, in order to perform PCNL surgery with balloon dilation. A balloon dilation catheter kit includes a balloon dilation catheter, a sheath, a two-way valve and an inflation device. A fascia dilation device set includes a puncture needle, a guidewire, dilators of different sizes, a sheath and a drainage catheter. Only one sheath and normally one dilator are needed during a surgery for creating a tract with balloon dilation. The remaining unused new sheath and several unused new dilators have to be discarded after the surgery.

Another problem with existing percutaneous nephrolithotomy lies in that it is very difficult for a physician to insert a dilator/balloon at a right distance. In use, in order to create a uniform channel without causing any unwanted damage to a patient, such a component needs to be put in the right place within a tissue. However, when inserting various components, it is often difficult to ensure the same insertion depth. Too much insertion may cause unwanted injury to the patient; too less insertion may lead to incomplete expansion. This increases the difficulty of surgery.

### SUMMARY

One aspect of the present disclosure is to provide a device set designed for percutaneous nephrolithotomy surgery, which set includes all necessary components to facilitate the physicians to use, and is advantageous in wastage and cost reduction.

A further aspect of the disclosure is that, with such a device set, the physician only needs to make sure that a first dilator is in the right place. The other dilators and balloon can follow the same insertion distance as the first dilator, which will greatly decrease the difficulty of surgery and increase the safety of surgery, thereby improving the reliability of surgery.

The present disclosure provides two kinds of device sets designed for PCNL surgery, respectively for the way of balloon catheter dilation and the way of dilator dilation. A device set designed for PCNL surgery using the way of balloon catheter dilation, comprising a puncture needle, a guidewire, a dilator, a balloon catheter, a sheath and a drainage catheter, can be called as a balloon catheter dilation device set. A device set designed for PCNL surgery using the way of dilator dilation, comprising a puncture needle, a guidewire, a plurality of dilators having different sizes, a sheath and a drainage catheter, can be called as a fascia dilation device set.

In either of the above two sets, all the included components can be placed in a single package. A plurality of compartments, for example in the form of recesses, are formed in the package, for accommodating stably the components to prevent the components from any damage caused by colliding with each other during handling.

For the balloon catheter dilation device set, the dilator has a tip length that is substantially the same as a tip length of the balloon catheter; for the fascia dilation device set, all the dilators have substantially the same tip length. Herein, a tip length of the dilator refers to a length of a distal cone portion of the dilator, and a tip length of the balloon catheter refers to a length of a portion between a distal tip of the balloon catheter and a shoulder of a distal cone of the balloon catheter.

By arranging such that the tip lengths are substantially the same, the above-mentioned another problem can be solved, and the above-mentioned another object of the present disclosure can be achieved. The above-mentioned problem arises because, in the existing products, the tips of the dilator and the balloon catheter have different lengths, while the tip portions are non-effective for the dilation of the channel (these portions can be called as a non-effective portion). In use, the insertion of various components often results in inconsistent insertion due to the presence of the tips.

The tip length may be in a range of 0.8 cm to 3 cm, for example 1.2 cm or 2 cm.

In either of the above two device sets, one or more of the puncture needle, guidewire, dilator, balloon catheter, sheath and drainage catheter are provided with scaler markers.

In the above balloon catheter dilation device set, the sheath has a size matched with a size of the balloon catheter; a size of the drainage catheter is 2Fr less than a size of the balloon catheter.

The balloon catheter dilation device set may further comprise a drainage bag, an inflation device, a stopcock, and/or a securement device. Preferably, the drainage bag may have a volume in a range of 500 ml to 3000 ml.

The above balloon catheter dilation device set may be sized to be 12Fr, 14Fr, 16Fr, 18Fr, 20Fr, 22Fr, 24Fr, 26Fr, 28Fr, 30Fr, or 32Fr.

In the above fascia dilation device set, the sheath has a size matched with a size of the dilator having the largest size; a size of the drainage catheter is 2Fr less than a size of the dilator having the largest size.

The above fascia dilation device set may further comprise a drainage bag and/or a securement device. The drainage bag has a volume in a range of 500 ml to 3000 ml.

The above fascia dilation device set may be sized to be 8Fr, 10Fr, 12Fr, 14Fr, 16Fr, 18Fr, 20Fr, 22Fr, 24Fr, 26Fr, 28Fr, 30Fr, or 32Fr.

In either of the above two device sets, the sheath may be a common sheath or a tearable sheath.

Other objects, features, and details of the present disclosure can be more fully understood with reference to the following detailed description of exemplary embodiments in conjunction with the accompanying drawings and according to the appended claims. A person skilled in the art will understand advantages of corresponding embodiments and various additional embodiments by reading the following detailed description of the corresponding embodiments with reference to the drawings as listed below. In addition, features in the drawings discussed below are not necessarily drawn to scale. Sizes of the features and elements in the drawings may be appropriately enlarged or reduced to more clearly illustrate the embodiments of the present disclosure.

### BRIEF DESCRIPTION OF THE DRAWINGS

The present disclosure is further described as below with reference to the accompanying drawings and embodiments, wherein like reference numerals refer to like elements.

FIG. 1 is an overall schematic view of a balloon catheter dilation device set designed for percutaneous nephrolithotomy surgery according to a first embodiment of the present disclosure.

FIG. 2 is a schematic view of tips of the balloon catheter and of the dilator as shown in FIG. 1 according to the first embodiment of the present disclosure.

FIG. 3 is an overall schematic view of a fascia dilation device set designed for percutaneous nephrolithotomy surgery according to a second embodiment of the present disclosure.

FIG. 4 is a schematic view of a tip of the dilator as shown in FIG. 3 according to the second embodiment of the present disclosure.

### DETAILED DESCRIPTION

Various illustrative embodiments of the present disclosure are described below. In this description, for the sake of explanation only, various systems, structures and devices are schematically depicted in the drawings, but all the features of actual systems, structures and devices are not described. For example, well-known functions or structures are not described in detail to avoid unnecessary details to obscure the present disclosure. Of course, it should be understood that in any practical application, many specific implementation decisions need to be made to achieve the specific goals of the developer or user, and the system-related and industry-related restrictions need to be observed. These specific goals may vary with actual applications. In addition, it should be understood that although such specific implementation decisions are complicated and time-consuming, this is a routine task for those of ordinary skill in the art who benefit from the present application.

The terms and phrases used herein should be understood and interpreted as having a meaning consistent with the understanding of those terms and phrases by those skilled in the relevant art. The consistent usage of terms or phrases herein is not intended to imply a specific definition of the term or phrase, that is, a definition that differs from the ordinary and customary meanings understood by those skilled in the art. For terms or phrases intended to have a special meaning, that is, meanings different from those understood by the skilled person, this special meanings will be clearly listed in the description by definition, giving special meanings to the term or phrase directly and unambiguously.

Unless the content requires, throughout the following description and claims, the words “comprising” and its variations, such as “including”, will be interpreted in an open and inclusive sense, that is, as “comprising but not limited to”.

In the present disclosure, the terms “first”, “second”, and the like are used for descriptive purposes only and cannot be construed as indicating relative importance or indicating the number of technical features. Thus, features defined as “first”, “second”, or the like may explicitly or implicitly include one or more of the features. In the description of the present disclosure, the meaning of “a plurality of” is two or more, unless otherwise specified.

In the present disclosure, unless otherwise clearly specified and defined, such terms as “mounted”, “connected”, “coupled”, “connected”, “fixed”, and so on should be understood in a broad sense. For example, it can be a fixed connection, or a detachable connection, or an integral connection; it can be a mechanical connection or an electrical connection; it can be directly connected or be indirectly connected via an intermediate medium; or it can be an internal communication between two components. Those of ordinary skill in the art can understand the specific meanings of the above terms in the present disclosure according to specific situations.

In the present disclosure, for convenience of description, the operator (for example, a physician) is used as a reference object, and the side close to the operator is called “proximal”, and the side away from the operator is called “distal”.

### First Embodiment

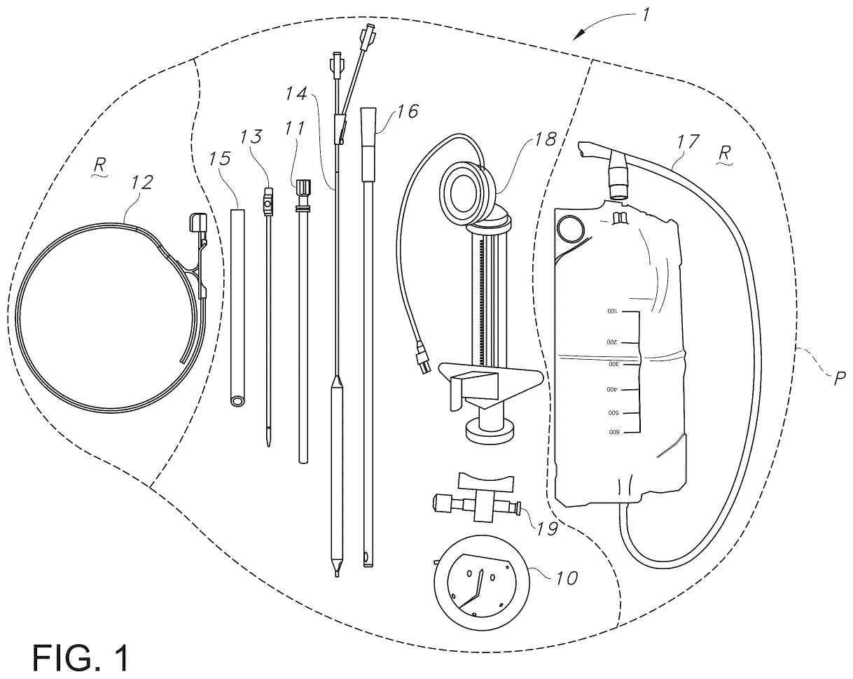

Referring to FIG. 1, the first embodiment according to the present disclosure provides a balloon catheter dilator device set 1 designed for the percutaneous nephrolithotomy surgery.

The balloon catheter dilator set 1 includes or consists of the following components:
a) 1pcs 18G puncture needle 11;
b) 1pcs 0.035″ or 0.038″ guidewire 12;
c) 1pcs 8Fr dilator or 1pcs 10Fr dilator (only an 8Fr dilator 13 is shown in the figure);
d) 1pcs balloon catheter 14 having a size the same as a set size of the device set;
e) 1pcs sheath 15 adapted to match with the size of the balloon catheter 14;
f) 1pcs drainage catheter 16 having a size that is 2Fr less than the size of the balloon catheter 14;
g) 1pcs drainage bag 17 having a volume in a range of 500 ml to 3000 ml;
h) 1pcs inflation device 18;
i) 1pcs stopcock 19; and
j) 1pcs securement device 10.

The set size of the balloon catheter dilation device set 1 may include 12Fr, 14Fr, 16Fr, 18Fr, 20Fr, 22Fr, 24Fr, 26Fr, 28Fr, 30Fr, or 32Fr. The set size thereof is an inner diameter of a desired nephrostomy tract (i.e., a stone removal path) that is finally completed.

The balloon catheter 14 has the same specification or nominal size as the set size. For example, as shown in FIG. 1, the balloon catheter dilator device set 1 may have a set size of 24 Fr, in which case it may be referred to as a 24Fr balloon catheter dilator device set. In the 24Fr balloon catheter dilator device set, the specification or nominal size of the balloon catheter 14 is 24Fr, the size of the sheath 15 is 24Fr, and the size of the drainage catheter 16 is 22Fr; and the dilator may be selected to be an 8Fr dilator (as shown in FIG. 1) or a 10Fr dilator. Physicians can select to use the balloon catheter dilator device sets of different sizes according to patients, conditions, etc.

The device set may include all of the above components, or some of the above components.

The components included in the device set can be placed in a single package P. A plurality of compartments, for example in the form of recesses R, are formed in the package, and can be configured for accommodating stably various components to prevent the components from any damage caused by colliding with each other during handling.

Except for the stopcock and the securement device, all the other components may be provided with scale markers, so as to facilitate the physician to get to know the usage status of the components at any time during use, such as insertion depth, filled volume, inflation pressure, etc.

The sheath may be a common sheath or a tearable sheath.

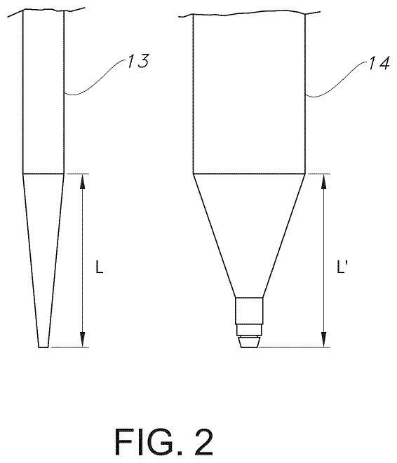

In particular, in the balloon catheter dilation device set 1, the tip length L of the dilator 13 is substantially the same as the distal tip length L′ of the balloon catheter 14. Herein, as shown in FIG. 2, a tip length of the dilator 13 refers to a length of a distal cone portion of the dilator, and a tip length of the balloon catheter 14 refers to a length of a portion between a distal tip of the balloon catheter and a shoulder of a distal cone of the balloon catheter. The tip length L and L′ may be in a range of 0.8 cm to 3 cm, for example 1.2 cm or 2 cm. The tip length L of the dilator and the tip length L′ of the balloon catheter have a difference less than 4 mm, and can be regarded as being substantially the same length.

During surgery, physician can insert a puncture needle 11 as guided with x-ray and/or ultrasound fluoroscopy. After positioning the puncture needle in place, the physician can record its insertion depth. Then, as guided by the guidewire 12 and with the assistance of scale markers, for example, an 8Fr dilator or a 10Fr dilator is inserted, with a dilator insertion depth equal to the puncture needle's insertion depth plus the tip length (for example, the tip length is 1.2 cm). The insertion depth of the subsequently inserted balloon catheter 14 is the same as the insertion depth of the dilator 13.

This device set solves the aforementioned technical problems. In particular, physicians do not need to purchase several packages in order to get all required components, but only need to purchase one set to get ready for all the necessary components, which is very convenient for physicians. Moreover, all the components in the device set are needed during surgery, which will not cause wastage.

In addition, the tips of the dilator and of the balloon catheter in the device set of the present disclosure are designed to have substantially the same length, such that the physicians can easily insert the balloon catheter at the correct distance during use. This greatly reduces the difficulty of surgery, avoids undesirable injuries to the patient, and can ensure the reliability and safety of surgery.

### Second Embodiment

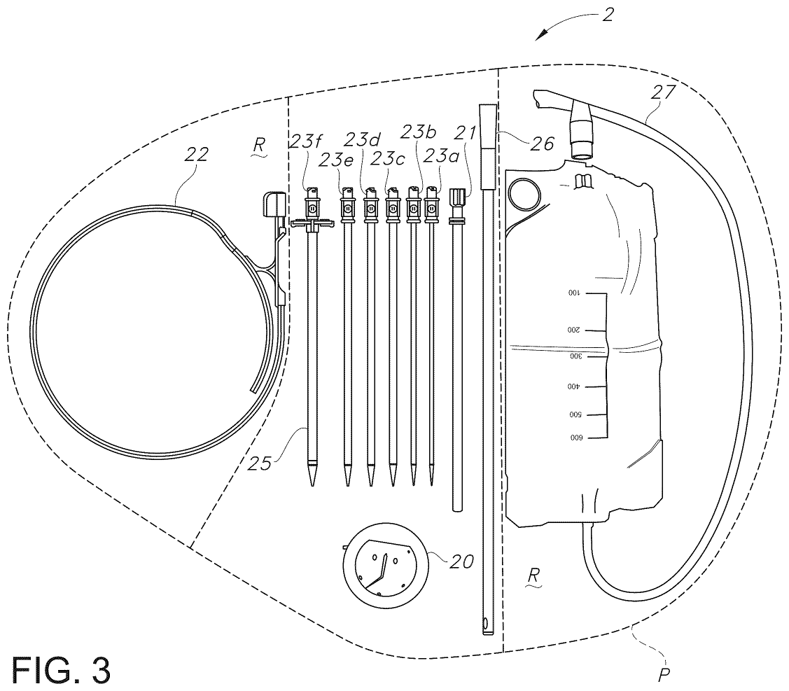

Referring to FIG. 3, the second embodiment according to the present disclosure provides a fascia dilation device set 2 designed for the percutaneous nephrolithotomy surgery.

The fascia dilation device set 2 includes or consists of the following components:
a) 1pcs 18G puncture needle 21;
b) 1pcs 0.035″ or 0.038″ guidewire 22;
c) a plurality of dilators 23a-23f having different sizes, the dilators having a number and sizes dependent on the set size of the device set;
d) 1pcs tearable sheath 25 having a size matched with a dilator 23f having the largest size;
e) 1pcs drainage catheter 26 having a size 2Fr less than the dilator 23f having the largest size;
f) 1pcs drainage bag 27 having a volume in a range of 500 ml to 3000 ml; and
g) 1pcs securement device 20.

The set size of the fascia dilation device set 2 may include 8Fr, 10Fr, 12Fr, 14Fr, 16Fr, 18Fr, 20Fr, 22Fr, 24Fr, 26Fr, 28Fr, 30Fr, or 32Fr. The set size thereof is an inner diameter of a desired nephrostomy tract (i.e., a stone removal path) that is finally completed. The size of the dilator 23f having the largest size is the same as the set size of the device set. For example, as shown in FIG. 3, the fascia dilation device set 2 may have a set size of 18Fr, in which case it may be referred to as an 18Fr fascia dilation device set. Herein, the size of the dilator 23f having the largest size is 18Fr, the size of the sheath 25 is 18Fr, and the size of the drainage catheter 26 is 16Fr; and the sizes of the other dilators 23a to 23e are 8Fr, 10Fr, 12Fr, 14Fr, 16Fr in order (in the embodiment shown in FIG. 3, there are six dilators in total, in which the largest size dilator 23f is nested in the sheath 25). Physicians can select to use various balloon catheter dilator device sets of different set sizes according to different patients, different conditions, etc.

The device set may include all of the above components, or some of the above components.

Similar to the first embodiment, the components included in the device set according to the second embodiment can be placed in a single package P. A plurality of compartments, for example in the form of recesses R, are formed in the package, and can be configured for accommodating stably various components to prevent the components from any damage caused by colliding with each other during handling.

Except for the guidewire and the securement device, all the other components may be provided with scale markers, so as to facilitate the physician to get to know the usage status of the components at any time during use, such as insertion depth, filled volume, inflation pressure, etc.

In this embodiment, the sheath is shown to be a tearable sheath. Of course, a non-tearable common sheath may also be used.

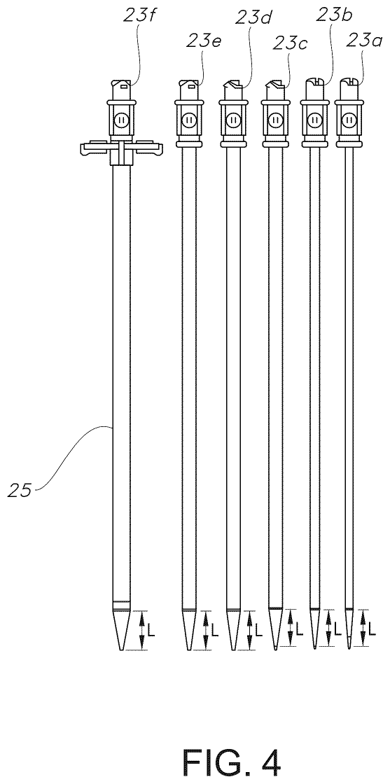

In particular, in the fascia dilation device set, the tip lengths L of all the dilators 23a to 23f are substantially the same, as shown in FIG. 4. Herein, a tip length L of the dilator refers to a length of a distal cone portion of the dilator. The tip length L may be in a range of 0.8 cm to 3 cm, for example 2 cm. The tip lengths of different dilators have a difference less than 4 mm, and can be regarded as being substantially the same length.

During surgery, physician can insert a puncture needle 21 as guided with x-ray and/or ultrasound fluoroscopy. After positioning the puncture needle 21 in place, the physician can record its insertion depth. Then, as guided by the guidewire 22 and with the assistance of scale markers, the dilators 23a to 23f are inserted in ascending order, with the insertion depth equal to the puncture needle 21's insertion depth plus the tip length (for example, the tip length is 2 cm).

Like the first embodiment, the device set according to the second embodiment solves the aforementioned technical problems existing in the prior art and produces advantageous technical effects, which will not be repeated here.

The present disclosure may include any feature or combination of features implicitly or explicitly disclosed herein or a generic concept thereof, and is not limited to any defined scope as listed above. Any elements, features and/or structural arrangements described herein may be combined in any suitable manner.

The specific embodiments disclosed above are merely exemplary, and it will be apparent to those skilled in the art who benefit from the teachings herein that the present disclosure can be modified and implemented in different but equivalent manners. For example, steps of the above-mentioned method can be performed in a different order. Further, the details of construction or design shown herein are not limited except those as set forth in the following claims. It is therefore obvious that changes and modifications can be made to the specific embodiments as disclosed above, and all these variations are considered to fall within the scope and spirit of the present disclosure. Therefore, the protection sought herein is set forth in the appended claims.

What is claimed is:

1. A device set designed for PCNL surgery, comprising a puncture needle, a guidewire, a plurality of dilators having different sizes, each of said plurality of dilators comprising a cone at a distal end of each respective dilator of the plurality of dilators, a sheath, and a drainage catheter which are all placed in a single package, wherein all the dilators have the same tip length from a proximal end of the cone to the distal end of each respective dilator.

2. The device set according to claim 1, wherein the tip length is in a range of 0.8 cm to 3 cm.

3. The device set according to claim 2, wherein the tip length is 2 cm.

4. The device set according to claim 1, wherein a plurality of chambers are formed in the package.

5. The device set according to claim 1, wherein one or more of the puncture needle, the guidewire, the dilators, the sheath, and the drainage catheter are provided with scale markers.

6. The device set according to claim 1, wherein the sheath has a size corresponding to a size of one of the dilators having the largest size.

7. The device set according to claim 1, wherein the sheath of the device set is a tearable sheath.

8. The device set according to claim 1, wherein a size of the drainage catheter is 2 Fr less than the size of one of the dilators having the largest size.

9. The device set according to claim 1, further comprising a drainage bag.

10. The device set according to claim 1, further comprising a drainage bag having a volume in a range of 500 ml to 3000 ml.
